(19) United States
(12) Patent Application Publication
(10) Pub. No.: US 2009/0069790 A1
(43) Pub. Date: Mar. 12, 2009

(54) SURFACE PROPERTIES OF POLYMERIC MATERIALS WITH NANOSCALE FUNCTIONAL COATING

(76) Inventors: Edward Maxwell Yokley, Anderson, SC (US); Yaw Samuel Obeng, Vienna, VA (US)

Correspondence Address: HITT GAINES P.C., P.O. BOX 832570, RICHARDSON, TX, 75083, US

(21) Appl. No.: 12/206,013

(22) Filed: Sep. 8, 2008

Related U.S. Application Data
(60) Provisional application No. 60/970,582, filed on Sep. 7, 2007.

(51) Int. Cl. A61M 25/00; B05D 3/06; B32B 27/36; B32B 27/32

(52) U.S. Cl. 604/523; 427/536; 428/394; 428/522; 428/523

(57) ABSTRACT

A method of manufacturing a polymeric object that comprises providing a polymeric substrate, and exposing said substrate to a first stage that includes an initial plasma reactant so as to reduce a water contact angle of a surface of the substrate, and, wherein the initial plasma treatment activates the surface to a grafting reaction, The method further includes exposing the activated substrate surface to a second stage that includes a second plasma reactant to thereby deposit a grafted material on the activated substrate surface to form a grafted surface.

### CROSS-REFERENCE TO RELATED APPLICATION

This Application claims the benefit of U.S. Provisional Application Ser. No. 60/970,582 filed on Sep. 7, 2007, entitled “IMPROVING SURFACE PROPERTIES OF POLYMERIC MATERIALS WITH NANOSCALE FUNCTIONAL COATING,” commonly assigned with the present invention and incorporated herein by reference.

### TECHNICAL FIELD OF THE INVENTION

The invention is directed, in general, polymeric objects and more particularly, to surface modification of objects, and methods of manufacturing thereof.

### BACKGROUND OF THE INVENTION

There is a need for engineering the air-polymer interface for specific applications. For example, it is often desirous modify the surface without altering the bulk properties of substrates and nanoscale coatings have the potential to greatly enhance the structural and functional performance of fabricated polymeric devices. Enhancement of the surface can occur with designed organic, inorganic or hybrid polymeric coatings. Traditional coating techniques such as spraying, painting, dip- or spin coating have proved difficult and unreliable due both to the properties of the coatings, and the typically low surface energy of the substrates.

Plasma surface modifications are used to produce nanoscale grafted organic and ceramic coatings rapidly and reproducibly. Application specific surface alterations are provided for by appropriate secondary grafting conditions that are compatible with the subsequent use of the device.

A group of invasive medical devices, feeding tubes, catheters, stents, needles and orthoscopic surgery tubes are in wide use that provide benefits relative to more extensive surgical procedures, outpatient self care and management, and unique treatments. However, these devices have long been known to cause injury, damage and discomfort to patients. The origins of the damage and discomfort problems stem from adhesion to tissues and tearing during insertion and removal, and inflammation and infection development during implantation. Adhesion failure of the coatings is a typical failure mode and results in the de-bonding of relatively large sections of the coatings on the cellular scale, leading to irritation, inflammation, pain, and a variety of local tissue responses that range from benign to life threatening to the patient.

The wide use of polymeric objects for skin and tissue contact applications has been limited by several confounding requirements. The objects need to be inert, non-toxic, and stable in the biological system. In general the materials that meet these requirements have low surface energy and tend to be hydrophobic. Examples of such materials are polyethylenes, polypropylenes, ABS, polycarbonates, and silicones. In the cases where natural polymers, such as latex rubbers are used in the construction of the object or device, there is the further complication of sensitivity to the natural rubber proteins and the materials used in the vulcanization of the natural latex.

In the cases where the objects are invasive, or function by motion at the skin surface, much work has been directed toward modification of the surfaces to deal with discomfort and tissue damage caused by the sliding of these inert surfaces over wet tissues. There is a wide literature in the design of surface coatings to mitigate these harmful effects to the patient or user. These devices are in wide use because they provide benefits relative to more extensive surgical procedures, outpatient self care and management, and unique treatments. The origins of the damage and discomfort problems stem from adhesion to tissues and tearing during insertion and removal, and inflammation and infection development during implantation. The industry has responded to these needs with the development of lubricious dip coatings and coatings that elute drug entities from the surface. These coatings are an improvement, but they generally suffer from poor adhesive bonding to the underlying surface. The device materials of choice are inherently non reactive to reduce the incidence of reactions with the surrounding tissues, and as a result tend to be materials with low surface energy and poor moisture interaction.

The current state of the art method for surface modification of medical devices tends to be comprised of some surface activation (e.g., thermal, corona, electromagnetic wave irradiation (UV), air-constituent gas plasma) followed by exposure to a solution of the polymer to be adhered to the substrate. This method and the products produced by it are inherently inferior to what is theoretically possible. Specifically, the films tend to adhere poorly; they tend to be defective allowing small molecules and microbes to permeate them. Also the film tends to be non-uniform due to agglomeration of the polymers in solution. In some cases, the substrate swells and deforms over time. The dip coating route also limits the kinds of molecules that can be put on the devices to solvent soluble species, which exclude most inorganic materials. The solvent based coatings also require capital intensive solvent removal and drying steps on devices made of multiple materials with irregular shapes.

Previous methods to achieve such surface coatings are deficient in their delamination performance and are both capital intensive and difficult to apply. There are capital requirements for solvent removal, and process control issues for urethane type reactive coating processes when implemented on large scale. Further, there are difficulties in the subsequent sterilization of the devices, since some of the coating chemistries are not compatible with autoclaving, Ethylene Oxide sterilization, or photochemical/radiation methods.

Traditional coating techniques have proved difficult to apply due both to the properties of the coatings, and the typically low surface energy of the preferred substrates. The substrates of choice are typically polyolefin, styrenic, silicone, vulcanized and natural rubbers based materials with non-polar surfaces without reactive functional groups to reduce tissue adhesion and interaction. In addition, these polymers have low melt temperatures that are incompatible with high temperature coating processes. The substrate materials have low surface energy and are resistant forming good adhesive bonds to most types of functional coatings.

There is therefore a need in the art to develop methods, processes and materials to address these deficiencies.

### SUMMARY OF THE INVENTION

To address the above-discussed deficiencies of the prior art, one embodiment of the invention provides a implantable polymeric object made by a process. The process comprises providing a polymeric substrate, and exposing the substrate to an initial plasma reactant so as to reduce a water contact angle of a surface of said substrate. The initial plasma treatment activates the surface to a grafting reaction. The process further includes exposing the activated substrate surface to a second plasma reactant to thereby deposit a grafted material on the activated substrate surface to form a grafted surface, The second plasma reactant includes a reactive precursor for the grafted material, and the initial plasma reactant and said second plasma reactant are generated in a plasma chamber having electrodes. The electrodes are maintained in a range from about 10° C. to about 100° C.

Other embodiments made by the above-described process include a polymeric object configured for external skin contact, a polymeric fiber and a water-resistant and abrasion-resistant polymeric device.

Still another embodiment is a method of manufacturing a polymeric object. The method comprises providing a polymeric substrate, and exposing said substrate to a first stage that includes an initial plasma reactant so as to reduce a water contact angle of a surface of the substrate, and, wherein the initial plasma treatment activates the surface to a grafting reaction, The method further includes exposing the activated substrate surface to a second stage that includes a second plasma reactant to thereby deposit a grafted material on the activated substrate surface to form a grafted surface.

### BRIEF DESCRIPTION OF THE DRAWINGS

For a more complete understanding of the invention, reference is now made to the following descriptions taken in conjunction with the accompanying drawings, in which:

FIGS. 1A-1C present perspective views of an example polymeric object of the present invention at different stages of fabrication.

### DETAILED DESCRIPTION

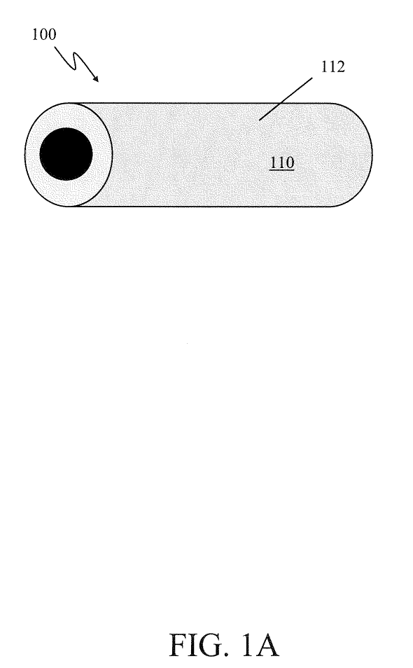

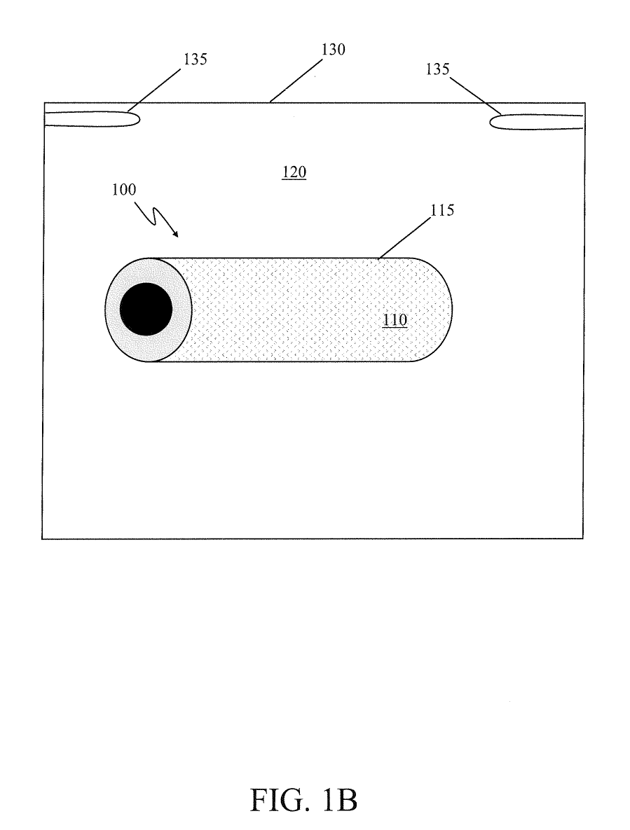

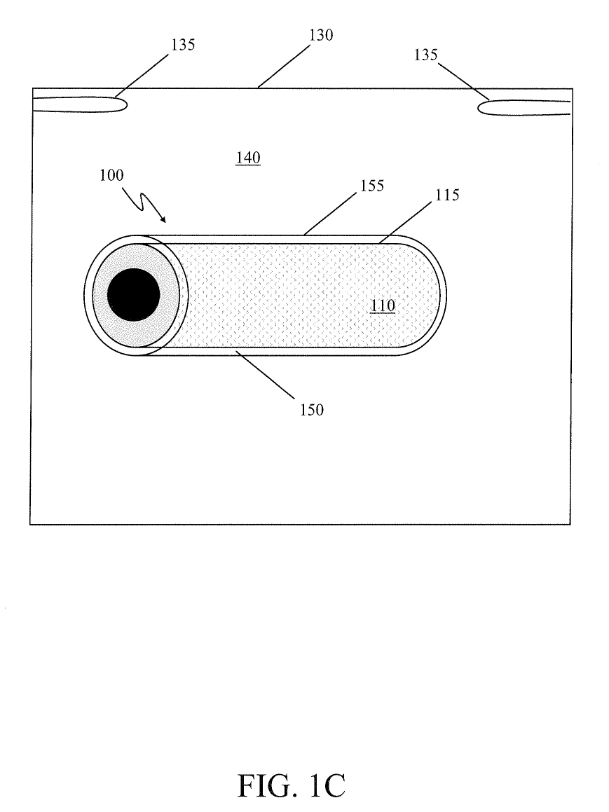

FIGS. 1A-1C presents perspective views of an example polymeric object 100 of the present invention at different stages of fabrication. The example embodiment presented is an implantable polymeric object 100 such as a catheter. As shown in FIG. 1A the object 100 is made by a process that comprises providing a polymeric substrate 110 having a surface 112.

FIG. 1B shows the object 100 while exposing the substrate 110 to an initial plasma reactant 120. The initial plasma treatment activates the substrate's surface 112 (FIG. 1A) to a grafting reaction. Such activation can be verified by measuring a reduction in a water contact angle of a surface 115 of the substrate 110 as compared to the surface 112 before the initial plasma treatment. As shown in FIG. 1B, the initial plasma treatment is carried out in a plasma chamber 130 having electrodes 135. The electrodes 135 are maintained in a range from about 10° C. to about 100° C. during the initial plasma treatment.

FIG. 1C shows the object 100 while exposing the activated substrate surface 115 to a second plasma reactant 140 to thereby deposit a grafted material 150 on the activated substrate surface 115 to form a grafted surface 155. The second plasma reactant 140 includes a reactive precursor (not show) for the grafted material 150, and the initial plasma reactant and said second plasma reactant are generated in the same plasma chamber 130 and using the same electrodes 135 as used for the initial plasma treatment. The electrodes 135 are maintained in a range from about 10° C. to about 100° C. during the exposure to the second plasma reactant 140.

Presented below are examples of how the above-described process could be implemented for particular embodiments of polymeric objects

We disclose here a process that both activates and coats irregular inert polymeric surfaces and operates on a dry-in, dry-out, sterile-out basis. We have used plasma based technology on a variety of polymeric and inorganic substrates to produce nanoscale grafted organic and ceramic coatings rapidly and reproducibly. Suitable precursors for surface modification include but not limited to organic monomers such as Allyl Alcohol, Allyl Amine, Vinyl Acetate, and Acrylic Acid., as well as Inorganic/Ceramic Monomers such as Tetraethoxyorthosilicate and Tetraisopropyltitanate TYZOR® TPT (DuPont).

Ideally, the substrates were treated in a two-phase inert gas plasma grafting process, comprised of a first phase which serves to activate the surface, followed by a second phase where reactive organic, or ceramic precursor monomers are introduced into a modified plasma environment. There is no upper limit to the number post surface activation steps and hence the number of layers of different materials that con be grafted to the substrate. Furthermore, the process is compatible with a wide range of both substrate materials and monomer chemistry types

The surface characteristics of the treated objects were sensitive to both the plasma conditions and secondary monomer reactant. The required temporal stability of the wettable surface and the subsequent uses of the items determine the specific sequence of treatments (plasma conditions and monomers used). When short lifetimes of less than a day are required, then appropriate single stage plasma conditions are the only concern. When permanent surface alteration is required, then appropriate secondary chemistry and grafting conditions are employed that are compatible with the subsequent use of the device. Properties such as the lubricity, micro-hardness or specific chemical reactivity of the surface to be bonded can be matched, through tuning with the mechanical properties of the coatings.

These multi-step plasma coating concepts have been adapted to modify thermoplastics. We have permanently grafted various vinyl, acrylic and pre-ceramic monomer to the surfaces of both polypropylenes and PE-VA foam surfaces. These modified surfaces were characterized by physical, microscopic and spectroscopic methods, and found to be composed of nanoscale domains of the coated material conformally bonded to the inert bulk or foam substrate.

The applicants have successfully adapted the 2-step plasma grafting technique described above into a multiple step process specifically designed for modifying and functionalizing the surfaces of medical devices. An advantage of the method described in this application is the ability to apply coatings on a dry-in dry-out basis in a sterile anaerobic environments. Using this method, parts can be placed into a treatment chamber dry and emerge after treatment both dry and sterile. The thin film coatings produced by the disclosed techniques are chemically bonded to the surface and are thus highly resistant to adhesion failures, delamination, flaking or debonding. The films are also coherent and uniform and are resistant to decohesion and tearing. Areas of the coated devices that need to remain non lubricious can be easily masked during the plasma coating process. The lubricity of the coating is activated by treatment of the surface with water or body fluids.

The so-deposited film stack could be comprised of organic and or inorganic polymers. The organic polymers are made from monomers can be selected of a group comprising, but not limited to, common lubricious monomers such as N-vinylpyrrolidinone and hydroxyethylmethacrylate and their copolymers ethylene and propylene oxide and their derivatives. The polymers are created in-situ at the substrate surface from treatment of the substrate in the plasma/monomer environment.

These plasma created polymer coatings provide lubricity when contacted with water or saline solution. The coated device is dry to the touch prior to water treatment for facile handling by medical or surgical personnel. The mechanical properties of the coatings, such as the flexibility of the deposited coating, can be modified by incorporation during the plasma polymerization of volatile crosslinking agents such as diallylethers, polyallylamines, gylcoldiacrylates or glycoldimethacrylates into the monomer stream.

Monomers with reactive functional groups containing amine, hydroxyl, and carboxylic acid functional groups can provide sites for the further coupling of surface binding or other materials and polymers, including designer drugs for targeted delivery. For example, known lubricious urethane polymers can be attached to preceding layers containing these reactive surfaces. Further, direct attachment or binding of gel mixtures of antibiotic or other drugs can be accomplished using standard solution coating or gas phase under non-plasma vacuum/reduced pressure techniques.

Some embodiments of the object of this invention include an invasive medical device such as a catheter, arthroscopic device or a rubberized coating on such a device. Some embodiments of the object are designed for external skin contact. Examples include gloves, condoms or skin stimulators, shoes, clothing items, or elastic bands in clothing items. In some cases, a hydrophilic surface is formed on the vulcanized latex object. This makes the object resistant to tearing, reduces skin irritation, discomfort to a wearer of the object, and reduces damage to biological tissues in contact with the object.

The specific conditions used during the initial plasma treatment can strongly influence characteristics of the polymeric substrate's surface. For instance, different initial plasma treatments followed by the same secondary plasma treatment can result in surfaces that are either hydrophilic or hydrophobic.

The initial plasma treatment can include a plasma reactant such as Helium, Argon, Nitrogen, Neon, Silane, Hydrogen and Oxygen and mixtures thereof. In some cases, the initial plasma treatment reaction is conducted at a radiofrequency power of 30 to 500 Watts.

The second plasma treatment can have secondary plasma reactants that include vinyl or acrylic monomers. Non-limiting example monomers include monomers 1-Vinyl-2-pyrrolidinone, 2-Hydroxyethylmethacrylate, Allyl Alcohol, Allyl Amine, Substituted Allyl Amines of 4-10 Carbon Atoms, Acrylic Acid, Acrylic Esters of 2-10 Carbon Atoms, Acrylamides of 3-10 Carbon Atoms. In some cases the resulting surface can be used as a glue layer under a conventional solvent, spray, dip or powder coating. The conventional coating is then used to bind a drug or other therapeutic material.

In other cases, the second plasma treatment can have secondary plasma reactants that include metal alkoxide esters of Silicon, Titanium, Aluminum, Zirconium, or Zinc.

A multiplicity of the plasma treatment process steps can be used to form multilayer coatings. In some cases, the initial and second plasma treatment is repeated 2 to 4 times. In some cases the first layer of a multilayer coating is an inorganic metal oxide coating. The inorganic metal oxide of the coating can include Silicon oxides, Titanium oxides, Aluminum oxides, or Zirconium oxides prepared by the secondary plasma reaction of the corresponding metal alkoxide esters.

Surface modification is achieved by exposing the vulcanized latex object to at least a two stage process. The first stage exposes the substrate to a primary plasma reactant to create a modified surface. In the second stage this modified surface is exposed to a secondary plasma reactant to produce a grafted surface. The grafted surface can be designed to be hydrophilic and resistant to causing said tearing, irritation, discomfort or damage on a dry-in/dry-out basis.

In the following we catalog a series of new illustrative, but non limiting, embodiments, designed to further illustrate the range of the nano-coating technology provided for by this invention.

The coatings of provided for in this application can be applied on a dry-in/dry-out basis; the articles to be coated can be placed into a treatment chamber dry and emerge after treatment both dry and sterile. The coatings include common lubricious monomers such as N-vinylpyrrolidinone which provides lubricity when contacted with water or saline solution. The coated device is dry to the touch prior to water treatment for facile handling by medical or surgical personnel. Further, the demonstrated coating of monomers with reactive handles containing amine, hydroxyl, and carboxylic acid functional groups can provide sites for the surface binding of antibiotic or other drugs.

One embodiment is a formed polymeric object, such as vulcanized latex object, whose surface is modified for contact with biological tissues. The term vulcanized latex object as used herein refers to tubes, gloves, catheters, condoms and similar objects made from cross linked thermoset precursors or formed polymeric thermoplastic. Illustrative examples of suitable vulcanized latex objects include: polypropylene drinking straws, tygon tubing, Red Rubber Bard Urethral Catheter, Lexan Panels, Polycarbonate Panels, Silicone Medical Tubing, Epoxy/Graphite Cylinder, and Latex Gloves.

Another embodiment is a process for creating the above-described surfaces on the vulcanized latex object. The formed polymeric object is made by exposing the polymeric substrate to at least two plasma treatments. An initial plasma treatment creates a modified reactive surface on the substrate. A second plasma treatment produces a grafted surface thereon. The initial plasma treatment is done while controlling the temperature of a radio-frequency electrodes to about 10 to 100° C.

Another embodiment of the sequential plasma processing steps is the modification of specialty filtration membranes, fibers, fabrics and related devices for use in bioprocessing, semiconductor and other high value industrial processes. For example, in micro reactor technology where the configurations take advantage of high reactor surface to volume ratios to achieve specific surface binding of catalysts or other reaction modifiers require engineered filter or fibrous surfaces. In most cases the filter media are made from chemically inert and low surface energy materials such as polyethylene, polypropylene, other polyolefins or polysulfone. The coatings of this invention have the ability to directly and selectively modify the chemical properties of channels, micro-pinholes and tortuous paths of specific filters. The coatings of this invention are uniquely able to perform these operations since the activation is driven by the plasma which accesses all surfaces within the plasma reaction chamber, and the volatile monomers are delivered to the activated surface sites in the gas phase. Illustratively, the filter membrane can be modified to selectively bind specific ions and molecules. For example, by functionalizing the membrane with soft ligands (e.g., aliphatic, thiols, amines, etc.) one can selectively bind coinage metal ions (Ag+, Au+, etc).

The advantages of a rapid, general, and chemically flexible system that can be used on finished configurations on a dry-in/dry-out basis are clearly evident to those skilled in the art.

In yet another instance, the performance of poly-vinyl-acetate (PVA) polymer in aqueous environments is constrained by the difficulties in hydration, bio-fouling and lack of application specificity. Current art for addressing these concerns involves modifications to the composition of the aqueous environments. Such modifications are not always successful. The coatings of this invention have the ability to directly and selectively modify the chemical properties on surface of the PVA articles. The coatings of this invention are uniquely able to perform these operations since the activation is driven by the plasma which passive native unstable chemical structure on the surface of the PVA objects. Additionally, the volatile monomers delivered to the activated surface sites in the gas phase chemically modify the surface to selectively bind specific ions and molecules. For example, by functionalizing the membrane with soft ligands (e.g., aliphatic-, thiols, amines, etc.) enables the PVA objects to selectively bind metal ions (Ag+, Au+, Cu2+, etc). Specifically, PVA brushes modified by the coatings of this invention can bind detrimental metallic species, such as Cu-and other metallic ions, to proactively address yield and reliability limiting dielectric contamination.

Similarly, by appropriate choice of surface finishing chemistry the PVA object surface can allow or prevent binding of biological species. By enabling the adhesion of biological elements the modified PVA surface serve a scaffold for the growth of biological systems. Alternatively, by appropriate choice of such coatings, it is possible to also prevent the adhesion of biological systems to such modified surface, thus preventing bio-fouling of such objects.

Yet another embodiment of the current invention involves the retention of pigment on fabrics other similar substrates. Reactive titanium intermediates which will leave reactive titanium-centered binding sites were used to increase the attachment of the TiO2 pigment to cellulose fibers. The reactive titanium functionality was covalently bonded to the cellulose fibers, and served as ‘glue’ to the other pigment of interests. Alternatively, the Ti-centers were fully hydrolyzed and oxidized to afford uncreative terminal TiO2. In this embodiment, the reactive titanates, dissolved in a mixture of organic solvents and ligands fluids, were introduced in a dry gas stream and then fed into a plasma chamber activation The reactive handles were effectively introduced into the bulk cellulose pulp using reactive species, and subsequently reacted to form Ti-oxides/hydroxide nano-clusters.

Another embodiment of the current invention is the formation of protective and abrasion resistant surfaces. The effect of sand erosion and abrasion on helicopter rotors blades is a perennial problem in dusty or desert environments. The current art employs erosion protection in the form of sacrificial leading edge strips Increased durability of ceramic modified thermoplastics in corrosive aqueous abrasive environments have been demonstrated. In particular, the two stage plasma based coatings of this invention of has been used to coat SiOx and TiOx on both thermoplastic and thermoset materials. The coatings of this invention, in particular the inorganic oxides as applied to the polyurethane rotor protection boots can provide effective aqueous surface protection in a high abrasive environment to improve durability of the rotors.

In like manner protective, abrasion resistant coatings can be applied by these methods to polymeric materials such as polycarbonate, acrylics, carbon fiber composites or other thermoplastics without altering the integrity of the parts. Ceramic Si and Ti oxide coatings as previously described and Carbon coatings formed from hydrocarbon reduction or from plasma based decomposition of Carbon Monoxide/Hydrogen Synthesis Gas mixtures are particularly effective as abrasion resistant coatings.

In the following we describe the experimental details the inventions that enable embodiments detailed above. Tables 1 and 2 are compendia of experimental results, using the methods of this invention.

TABLE 1

| | | | P1 | | | | | | | | | | P3 | | | |
|---|---|---|---|---|---|---|---|---|---|---|---|---|---|---|---|---|
| | P1 | P1 | Pres- | P1 | | P2 | P2 | P2 | | P3 | P3 | P3 | Pres- | | | |
| Expt | Power/ | Time/ | sure/ | Gas | P2 | Power/ | Time/ | Pressure/ | P2 | Mono- | Power/ | Time/ | sure/ | P3 | | |
| # | Watts | mins | mTorr | Mix | Monomer | Watts | mins | mTorr | Gas | mer | Watts | mins | mTorr | Gas | DIWCA | pH |
| 1 | 0 | 0 | N/A | N/A | NVP | 50 | 20 | 275 | Ar | N/A | NA | N/A | N/A | N/A | 10 | |
| 2 | 100 | 15 | 250 | Ar | None | 50 | 20 | 250 | Ar | N/A | NA | N/A | N/A | N/A | 98 | |
| 3 | 50 | 10 | 250 | Ar | NVP | 50 | 20 | 250 | Ar | N/A | NA | N/A | N/A | N/A | 74 | |
| 4 | 50 | 15 | 250 | Ar | NVP | 50 | 20 | 250 | Ar | N/A | NA | N/A | N/A | N/A | 74 | |
| 5 | 100 | 15 | 250 | Ar | NVP | 50 | 20 | 250 | Ar | N/A | NA | N/A | N/A | N/A | 54 | |
| 6 | 100 | 15 | 250 | Ar | NVP | 50 | 20 | 250 | Ar | N/A | NA | N/A | N/A | N/A | 49 | |
| 7 | 100 | 15 | 250 | Ar | TEOS | 50 | 20 | 250 | Ar | N/A | NA | N/A | N/A | N/A | 60 | |
| 8 | 100 | 15 | 250 | Ar | HEMA | 50 | 20 | 250 | Ar | N/A | NA | N/A | N/A | N/A | 32 | |
| 9 | 50 | 5 | 350 | 20:80 | HEMA | 50 | 20 | 250 | Ar | N/A | NA | N/A | N/A | N/A | 85 | |
| | | | | O₂:Ar | | | | | | | | | | | | |
| 10 | 50 | 3 | 350 | 20:80 | Allylamine | 50 | 20 | 250 | Ar | N/A | NA | N/A | N/A | N/A | 74 | 8 |
| | | | | O₂:Ar | | | | | | | | | | | | |
| 11 | 100 | 4 | 350 | 20:80 | Allylamine | 50 | 20 | 250 | Ar | N/A | NA | N/A | N/A | N/A | 78 | |
| | | | | O₂:Ar | | | | | | | | | | | | |
| 12 | 100 | 15 | 250 | Ar | TEOS | 50 | 20 | 250 | Ar | HEMA | 50 | 20 | 250 | Ar | 10 | |
| 13 | 100 | 5 | 350 | 20:80 | TEOS | 50 | 20 | 250 | Ar | HEMA | 50 | 20 | 250 | Ar | 60 | |
| | | | | O₂:Ar | | | | | | | | | | | | |

TABLE 2

| | | P1 | P1 | | | | | | P2 | | |
|---|---|---|---|---|---|---|---|---|---|---|---|
| | | Power/ | Time/ | P1 Pressure/ | P1 Gas | | P2 Power/ | P2 Time/ | Pressure/ | | DIW |
| Expt # | Substrate | Watts | mins | mTorr | Mix | P2 Monomer | Watts | mins | mTorr | P2 Gas | CA |
| 14 | Tygon Tubing | 0 | 0 | N/A | N/A | NVP | 50 | 20 | 275 | Ar | 102 |
| 15 | Tygon Tubing | 50 | 15 | 350 | 20:80 O₂:Ar | TYZOR PTP | 50 | 20 | 250 | Ar | 62 |
| 16 | Tygon Tubing | 100 | 15 | 250 | 20:80 O₂:Ar | HEMA | 50 | 20 | 250 | Ar | 69 |
| 17 | Red Rubber | 100 | 15 | 250 | 20:80 O₂:Ar | HMDS | 50 | 20 | 250 | Ar | 70 |
| | Bard Urethral | | | | | | | | | | |
| | Catheter | | | | | | | | | | |

In table 1, the common illustrative substrate was polypropylene, while in table 2 both tygon tubing and red rubber Bard urethral catheter were used as non-limiting illustrative examples of substrates. In tables 1 and 2, HEMA is an acronym for 2-hydrozyethyl Methraylate, HMDS represents hexammethyldisilazane, NVP represents N-Vinylpyrrolinone TEOS represents tetraethylorthosilicate, and TYZOR TPT represents tetraethylorthotitanate or tetraisopropyltitanate respectively.

Three types of experiments were conducted: Experiments 1 and 14 involved a one-step coating in which the surface modification monomer (e.g., N-Vinylpyrrolidinone) was deposited on native polypropylene surface, without prior plasma conditioning. While the surface was made permanently hydrophilic, the coating was not uniform.

Experiments 2 through 11 and 15, through 17 involved a two-step plasma coating process where the substrate surface was first prepared with pure Ar or O2/Ar mixture plasma were also performed. This step, among other benefits, serves to clean the substrate.

Experiments 12 and 13 involved a three-step coating process where the substrates prepared in the 2-Step Coating processes are optionally further modified.

Experiments 1 through 17, demonstrate the process conditions suitable for the functionalizing polymer surfaces precursors, including common lubricious organic monomers such as N-vinylpyrrolidinone (NVP) and hydroxyethylmethacrylate (HEMA) and their copolymers, as well inorganic precursors such as TEOS and TYZOR PTP. These functionalized polymers provide a range of lubricity when exposed to water or saline solution. All the coated devices provided for by the current invention are dry to the touch prior to water treatment. The flexibility of the deposited coating method provided for by this invention is demonstrated by the fact that the chemical and mechanical properties of the surface modifications can be modified by incorporation during the plasma polymerization of volatile cross linking agents such as diallylethers, polyallylamines, gylcoldiacrylates or glycoldimethacrylates into the monomer stream.

The surface pH depends on the functional groups at the air-polymer interface. As illustrated by the results from Experiment 8, an allylamine grafted polypropylene surface is basic, with a pH of about 8. In contrast, an allyl-alcohol grafted polypropylene surface is acidic, with a pH of less than 7.

Experiments 10 and 11 specifically demonstrated coating of monomers with reactive handles containing amines to afford a chemically basic surface that can provide sites for the surface binding or other materials and polymers. Similar strategies have also been used, by appropriate choice of monomer chemistries to create surfaces funtionalized with hydroxyl, and carboxylic acid functional groups. Lubricious urethane polymers and the binding of gel mixtures of antibiotic or other drugs can be accomplished using standard solution coating techniques on the surfaces afforded by the funtionalized surfaces.

The efficacy of the surface preparation and activation in plasma step-1 the choice of process conditions, such as plasma-1 process pressure, time, power and gas mixture significantly affects the final result in different surface energy of the finished product. For example, in comparing the water contact angles on articles produced in experiments 8 and 9 respectively, higher power, longer process time and lower process pressures at the plasma-1 stage resulted in a lower contact angle surface in experiment 8.

The coatings of provided for in this application can be applied on a dry-in/dry-out basis; the articles to be coated can be placed into a treatment chamber dry and emerge after treatment both dry and sterile. The lubricity of the coating is activated by treatment of the surface with water. The coating polymers are chemically bonded to the surface by the treatment process and are thus highly resistant to adhesion failures, delamination, flaking or debonding. Areas of the coated devices that need to remain non lubricious can be easily masked during the plasma coating process.

The polymers are made from monomers can include common lubricious monomers such as N-vinylpyrrolidinone and hydroxyethylmethacrylate and their copolymers, ethylene and propylene oxide and their derivatives. The polymers are created at the substrate surface from treatment of the substrate in the plasma/monomer environment. These polymers provide lubricity when contacted with water or saline solution. The coated device is dry to the touch prior to water treatment for facile handling by medical or surgical personnel. The flexibility of the deposited coating can be modified by incorporation during the plasma polymerization of volatile cross linking agents such as diallylethers, polyallylamines, gylcoldiacrylates or glycoldimethacrylates into the monomer stream.

Further, this invention provides for coating of monomers with reactive handles containing amine, hydroxyl, and carboxylic acid functional groups can provide sites for the surface binding or other materials and polymers. For example, known lubricious urethane polymers can be attached to these reactive surfaces. Further, direct attachment or binding of gel mixtures of antibiotic or other drugs can be accomplished using standard solution coating techniques. Depending on the functional groups at the surface (e.g., amines, thiols, carbonyl, alcohol, etc.), metal ion clusters can be complexed or ion-exchanged onto the device purposes for antiseptic and anti-fouling purposes.

In all cases, the modified surfaces showed permanent improvements in their hydrophilic (reduced water contact angles relative to the untreated substrates).

Similar to the observations on the polypropylene substrates, all the modified surfaces in this group of objects also showed permanent improvements in hydrophilicity (reduced water contact angles relative to the untreated substrates).While various embodiments of the present invention have been shown and described herein, it will be obvious that such embodiments are provided by way of example only. Numerous variations, changes and substitutions may be made without departing from the invention herein.

Those skilled in the art to which the invention relates will appreciate that other and further additions, deletions, substitutions and modifications may be made to the described embodiments without departing from the scope of the invention.

What is claimed is:

1. An implantable polymeric object made by a process, comprising:
providing a polymeric substrate;
exposing said substrate to an initial plasma reactant so as to reduce a water contact angle of a surface of said substrate and wherein said initial plasma treatment activates said surface to a grafting reaction; and
exposing said activated substrate surface to a second plasma reactant to thereby deposit a grafted material on said activated substrate surface to form a grafted surface, wherein:
said second plasma reactant includes a reactive precursor for said grafted material, and
said initial plasma reactant and said second plasma reactant are generated in a plasma chamber having electrodes, said electrodes are maintained in a range from about 10° C. to about 100° C.

2. The object of claim 1, wherein said object is configured as a catheter for insertion into biological tissues.

3. The object of claim 2, wherein said grafted surface has a hydrophilic surface configured to prevent tearing, irritation, discomfort or damage to biological tissues.

4. The object of claim 1, wherein said initial plasma reactant is selected from a group consisting of: Helium, Argon, Nitrogen, Neon, Silane, Hydrogen and Oxygen and mixtures thereof.

5. The object of claim 1, wherein said reactive precursor is selected from a group consisting of: vinyl monomer, acrylic monomers and mixtures thereof.

6. The object of claim 1, wherein said reactive precursor is selected from a group consisting of: 1-Vinyl-2-pyrrolidinone, 2-Hydroxyethylmethacrylate, Allyl Alcohol, Allyl Amine, Substituted Allyl Amines of 4-10 Carbon Atoms, Acrylic Acid, Acrylic Esters of 2-10 Carbon Atoms, and Acrylamides of 3-10 Carbon Atoms.

7. The object of claim 1, wherein said reactive precursor is selected from a group consisting of: metal alkoxide esters of Silicon, metal alkoxide esters of Titanium, metal alkoxide esters of Aluminum, metal alkoxide esters of Zirconium, and metal alkoxide esters of Zinc.

8. The object of claim 1, wherein said deposition of said grafted material is repeated at least once to form a multilayer coating of said deposit a grafted material.

9. The object of claim 8, wherein a first layer of said multilayer coating is an inorganic metal oxide coating.

10. The object of claim 8, wherein said inorganic metal oxide coating is selected from the group consisting of: Silicon oxides, Titanium oxides, Aluminum oxides, and Zirconium oxides, and, wherein said reactive precursor corresponds to a metal alkoxide ester of said selected Silicon oxides, Titanium oxides, Aluminum oxides, or Zirconium oxides.

11. A polymeric object configured for external skin contact made by a process, comprising:
providing a polymeric substrate;
exposing said substrate to an initial plasma reactant so as to reduce a water contact angle of a surface of said substrate and wherein said initial plasma treatment activates said surface to a grafting reaction; and
exposing said activated substrate surface to a second plasma reactant to thereby deposit a grafted material on said activated substrate surface to form a grafted surface, wherein:
said second plasma reactant includes a reactive precursor for said grafted material, and
said initial plasma reactant and said second plasma reactant are generated in a plasma chamber having electrodes, said electrodes are maintained in a range from about 10° C. to about 100° C.

12. The object of claim 11, wherein said object is configured as one of: gloves, condoms or skin stimulators, shoes, clothing items, or elastic bands in clothing items.

13. The object of claim 11, wherein said initial plasma reactant is selected from a group consisting of: Helium, Argon, Nitrogen, Neon, Silane, Hydrogen and Oxygen and mixtures thereof.

14. The object of claim 11, wherein said reactive precursor is selected from a group consisting of: vinyl monomer, acrylic monomers and mixtures thereof.

15. The object of claim 1, wherein said reactive precursor is selected from a group consisting of: 1-Vinyl-2-pyrrolidinone, 2-Hydroxyethylmethacrylate, Allyl Alcohol, Allyl Amine, Substituted Allyl Amines of 4-10 Carbon Atoms, Acrylic Acid, Acrylic Esters of 2-10 Carbon Atoms, Acrylamides of 3-10 Carbon Atoms.

16. The object of claim 11, wherein said reactive precursor is selected from a group consisting of: metal alkoxide esters of Silicon, metal alkoxide esters of Titanium, metal alkoxide esters of Aluminum, metal alkoxide esters of Zirconium, metal alkoxide esters of Zinc and synthesis gas.

17. A polymeric fiber made by a process, comprising:
providing a polymeric fiber substrate;
exposing said fiber substrate to an initial plasma reactant so as to reduce a water contact angle of a surface of said fiber substrate and wherein said initial plasma treatment activates said surface to a grafting reaction; and
exposing said activated fiber substrate surface to a second plasma reactant to thereby deposit a grafted material on said activated substrate surface to form a grafted surface, wherein:
said second plasma reactant includes a reactive precursor for said grafted material, and
said initial plasma reactant and said second plasma reactant are generated in a plasma chamber having electrodes, said electrodes are maintained in a range from about 10° C. to about 100° C.

18. The polymeric fiber of claim 17, wherein said polymeric fiber is incorporated into a brush or a fabric.

19. The object of claim 17, wherein said initial plasma reactant is selected from a group consisting of: Helium, Argon, Nitrogen, Neon, Silane, Hydrogen and Oxygen and mixtures thereof.

20. The object of claim 17, wherein said reactive precursor is selected from a group consisting of: vinyl monomer, acrylic monomers and mixtures thereof.

21. The object of claim 17, wherein said reactive precursor is selected from a group consisting of: 1-Vinyl-2-pyrrolidinone, 2-Hydroxyethylmethacrylate, Allyl Alcohol, Allyl Amine, Substituted Allyl Amines of 4-10 Carbon Atoms, Acrylic Acid, Acrylic Esters of 2-10 Carbon Atoms, and Acrylamides of 3-10 Carbon Atoms.

22. The object of claim 18, wherein said reactive precursor is selected from a group consisting of: metal alkoxide esters of Silicon, metal alkoxide esters of Titanium, metal alkoxide esters of Aluminum, metal alkoxide esters of Zirconium, metal alkoxide esters of Zinc and synthesis gas.

23. A water-resistant and abrasion-resistant polymeric device made by the process, comprising:
providing a polymeric fiber substrate;
exposing said fiber substrate to an initial plasma reactant so as to reduce a water contact angle of a surface of said fiber substrate and wherein said initial plasma treatment activates said surface to a grafting reaction; and
exposing said activated fiber substrate surface to a second plasma reactant to thereby deposit a grafted material on said activated substrate surface to form a grafted surface, wherein:
said second plasma reactant includes a reactive precursor for said grafted material, and
said initial plasma reactant and said second plasma reactant are generated in a plasma chamber having electrodes, said electrodes are maintained in a range from about 10° C. to about 100° C.

24. The polymeric device of claim 23, wherein said initial plasma reactant is selected from a group consisting of: Helium, Argon, Nitrogen, Neon, Silane, Hydrogen and Oxygen and mixtures thereof.

25. The polymeric device of claim 23, wherein said reactive precursor is selected from a group consisting of: vinyl monomer, acrylic monomers and mixtures thereof.

26. The polymeric device of claim 23, wherein said reactive precursor is selected from a group consisting of: metal alkoxide esters of Silicon, metal alkoxide esters of Titanium, metal alkoxide esters of Aluminum, metal alkoxide esters of Zirconium, metal alkoxide esters of Zinc and synthesis gas.

27. A method of manufacturing a polymeric object, comprising:
providing a polymeric substrate;
exposing said substrate to a first stage that includes an initial plasma reactant so as to reduce a water contact angle of a surface of said substrate and wherein said initial plasma treatment activates said surface to a grafting reaction; and
exposing said activated substrate surface to a second stage that includes a second plasma reactant to thereby deposit a grafted material on said activated substrate surface to form a grafted surface.

28. The method of claim 27, wherein said initial plasma reactant is selected from a group consisting of: Helium, Argon, Nitrogen, Neon, Silane, Hydrogen and Oxygen and mixtures thereof.

29. The polymeric device of claim 27, wherein said reactive precursor is selected from a group consisting of: vinyl and acrylic monomers, silicate esters, titanate esters, aluminate esters and synthesis gas.

29. The polymeric device of claim 27, wherein said reactive precursor is selected from a group consisting of: 1-Vinyl-2-pyrrolidinone, 2-Hydroxyethylmethacrylate, Allyl Alcohol, Allyl Amine, Substituted Allyl Amines of 4-10 Carbon Atoms, Acrylic Acid, Acrylic Esters of 2-10 Carbon Atoms, and Acrylamides of 3-10 Carbon Atoms

30. The method of claim 27, wherein said first stage is conducted in a plasma chamber having electrodes maintained at in a range of about 10° C. to about 100° C.

31. The method of claim 27, wherein said first stage is conducted in a plasma chamber having electrodes transmitting a radiofrequency power of in a range of about 30 to about 500 Watts.

32. The method of claim 27, wherein said is conducted in a plasma chamber maintained at a pressure in a range of about 50 to 500 mTorr.

33. The method of claim 27, wherein said first stage and said second stage are sequentially repeated at least 2 times to thereby form a multilayered grafted material.

34. The method of claim 33, wherein a first iteration of said second stage uses said second plasma reactant that is a reactive precursor of inorganic metal oxide grafted material layer.

35. The method of claim 34, wherein said inorganic metal oxide is a Silicate or a Titanate.
